(19) United States
(12) Patent Application Publication
(10) Pub. No.: US 2024/0170125 A1
(43) Pub. Date: May 23, 2024

(54) DEVICE AND METHODS FOR MANAGING PHYSICAL EXERTIONS PREDICTED DURING A VR SESSION

(71) Applicant: SAMSUNG ELECTRONICS CO., LTD., Suwon-si (KR)

(72) Inventors: Ashish BHATT, Bangalore (IN); Muthukumaran NATARAJAN, Bangalore (IN); Kushlam CHOWRASIYA, Bangalore (IN); Aman Chetan FRAMEWALA, Bangalore (IN); Kaushik DAS, Bangalore (IN)

(73) Assignee: SAMSUNG ELECTRONICS CO., LTD., Suwon-si (KR)

(21) Appl. No.: 18/372,382

(22) Filed: Sep. 25, 2023

Related U.S. Application Data
(63) Continuation of application No. PCT/KR23/14173, filed on Sep. 19, 2023.

(30) Foreign Application Priority Data
Nov. 21, 2022 (IN) 202241068807

(51) Int. Cl. G16H 20/30; G06T 19/00; G16H 10/60; G16H 50/20

(52) U.S. Cl. CPC G16H 20/30; G06T 19/006; G16H 10/60; G16H 50/20

(57) ABSTRACT

According to an embodiment of the disclosure, a method performed by an electronic device for managing physical exertion of an user may include determining at least one physical strain exerted on at least one body part of the user in at least one Virtual Reality (VR) interaction of a VR session, identifying at least one real-world device where the at least one body part of the user performs at least one upcoming real-world activity subsequent to the at least one VR interaction, and altering at least one operational setting of the at least one identified real-world device, wherein the at least one operational setting corresponds to the at least one physical strain exerted on the at least one body part of the user.

### CROSS-REFERENCE TO RELATED APPLICATIONS

This application is a by-pass continuation application of International Application No. PCT/KR2023/014173, filed on Sep. 19, 2023, which is based on and claims priority to Indian Patent Application No. 202241066807, filed on Nov. 21, 2022, in the India Intellectual Property Office, the disclosures of which are incorporated by reference herein their entireties.

### BACKGROUND

### 1. Field

The disclosure relates to physical strains of an user due to interactions in Virtual Reality (VR) sessions and to mitigate repercussions of physical exertions predicted to occur during a VR session in Internet of Things (IOT) environment.

### 2. Description of Related Art

Virtual Reality (VR) games of a Head mounted display (HMD) provide players with valuable levels of physical exertions that may be much higher than their perceived exertions. Real-world devices like smart watches and heart-rate monitors may help in tracking the physical exertions during VR sessions. Currently, computer-implemented systems may identify the physical exertions using heart rates and stress levels and may provide inputs to an ongoing VR session to reduce the physical exertions in the VR world.

### SUMMARY

According to an embodiment of the disclosure, the method may include determining at least one physical strain exerted on at least one body part of the user in at least one Virtual Reality (VR) interaction of a VR session. According to an embodiment of the disclosure, the method may include identifying at least one real-world device where the at least one body part of the user performs at least one upcoming real-world activity subsequent to the at least one VR interaction. According to an embodiment of the disclosure, the method may include altering at least one operational setting of the at least one identified real-world device. According to an embodiment of the disclosure, the at least one operational setting corresponds to the at least one physical strain exerted on the at least one body part of the user.

According to an embodiment of the disclosure, an electronic device may include at least one memory storing at least one instruction and at least one processor operatively connected to the at least one memory and configured to execute the at least one instructions. According to an embodiment of the disclosure, at least one processor is configured to determine at least one physical strain exerted on at least one body part of the user in at least one Virtual Reality (VR) interaction of a VR session. According to an embodiment of the disclosure, at least one processor is configured to identify at least one real-world device where the at least one body part of the user performs at least one upcoming real-world activity subsequent to the at least one VR interaction. According to an embodiment of the disclosure, at least one processor is configured to alter at least one operational setting of the at least one identified real-world device. According to an embodiment of the disclosure, wherein the at least one operational setting corresponds to the at least one physical strain exerted on the at least one body part of the user.

These and other aspects of the embodiments herein will be better appreciated and understood when considered in conjunction with the following description and the accompanying drawings. The following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

Embodiments herein are illustrated in the accompanying drawings, throughout which like reference letters indicate corresponding parts in the various figures. The embodiments herein will be better understood from the following description with reference to the drawings, in which:

FIG. 1 depicts an example scenario where an user tries to pick up freshly-brewed coffee after a VR gaming session.

FIG. 2 depicts an example scenario where an user has a scheduled call with doctor after a VR meeting.

FIG. 3 depicts an example scenario where an user needs to remove washed clothes after a VR boxing activity.

FIG. 4 depicts an electronic device for managing physical exertion of an user predicted during a VR session, according to an embodiment of the disclosure as disclosed herein;

FIG. 5 depicts a system flow representation for managing physical exertion of an user predicted during a VR session, according to an embodiment of the disclosure as disclosed herein;

FIG. 6 depicts a detailed system flow representation for managing physical exertion of an user predicted during the VR session, according to an embodiment of the disclosure as disclosed herein;

FIG. 7 depicts a method for managing physical exertions of an user predicted during a VR session, according to an embodiment of the disclosure as disclosed herein;

FIG. 8 depicts a method for managing real-world activities of an user based on virtual world interactions of the user, according to an embodiment of the disclosure as disclosed herein;

FIG. 9A depicts a use case for managing physical exertion of an user by rescheduling a scheduled call after a long meeting in a VR session, according to an embodiment of the disclosure as disclosed herein;

FIG. 9B depicts a detailed functional representation of the device data aggregation module, according to an embodiment of the disclosure as disclosed herein;

FIG. 9C depicts a detailed functional representation of the VR context managing module, according to an embodiment of the disclosure as disclosed herein;

FIG. 9D depicts a detailed functional representation of the strain prediction module, according to an embodiment of the disclosure as disclosed herein;

FIG. 9E depicts eye strain prediction using the strain prediction module, according to an embodiment of the disclosure as disclosed herein;

FIG. 9F depicts throat strain prediction using the strain prediction module, according to an embodiment of the disclosure as disclosed herein;

FIG. 9G depicts a detailed functional representation of the strain activity correlation module, according to an embodiment of the disclosure as disclosed herein;

FIG. 9H depicts a detailed functional representation of the action recommendation module, according to an embodiment of the disclosure as disclosed herein;

FIG. 9I depicts a detailed functional representation of an action recommendation orchestrator, according to an embodiment of the disclosure as disclosed herein;

FIG. 10 depicts another use case for managing physical exertion of an user by delaying coffee preparation after a long gaming activity in a VR session, according to an embodiment of the disclosure as disclosed herein; and

FIG. 11 depicts another use case for managing physical exertion of an user by changing washing machine mode after a long gaming activity in a VR session, according to an embodiment of the disclosure as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Before undertaking the detailed description below, it may be advantageous to set forth definitions of certain words and phrases used throughout the present disclosure. The term “couple” and the derivatives thereof refer to any direct or indirect communication between two or more elements, whether or not those elements are in physical contact with each other. The terms “transmit”, “receive”, and “communicate” as well as the derivatives thereof encompass both direct and indirect communication. The terms “include” and “comprise”, and the derivatives thereof refer to inclusion without limitation. The term “or” is an inclusive term meaning “and/or”. The phrase “associated with,” as well as derivatives thereof, refer to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” refers to any device, system, or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of A, B, and C” includes any of the following combinations: A, B, C, A and B, A and C, B and C, and A and B and C, and any variations thereof. Similarly, the term “set” means one or more. Accordingly, the set of items may be a single item or a collection of two or more items.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as Read Only Memory (ROM), Random Access Memory (RAM), a hard disk drive, a Compact Disc (CD), a Digital Video Disc (DVD), or any other type of memory. A “non-transitory”computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Existing systems may collect stress data associated with an user of a VR device and determine a stress level of the user based on the stress data. Further, the systems may identify the stress level of the user and modify scenes in the VR world to reduce the stress level. Further, the systems may identify cyber sickness and may provide suggestions to the user for participating in different activities like exercises, taking breaks etc.

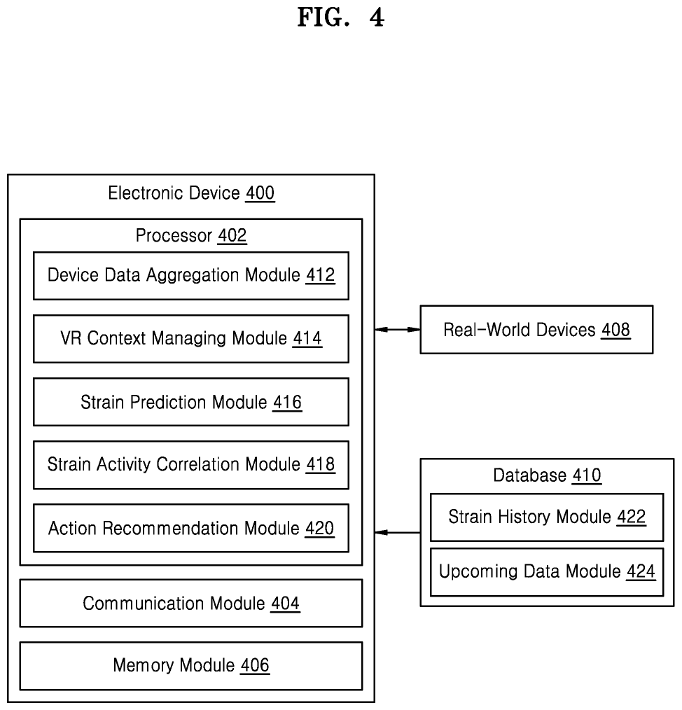

The embodiments herein provide a device and methods for monitoring one or more physical strains of an user due to interactions in a Virtual Reality (VR) session and recommending one or more personalized action suggestions for an upcoming physical activity in the real-world. In the drawings, and more particularly, FIGS. 4 through 11, similar reference characters denote corresponding features consistently throughout the figures, there are shown embodiments.

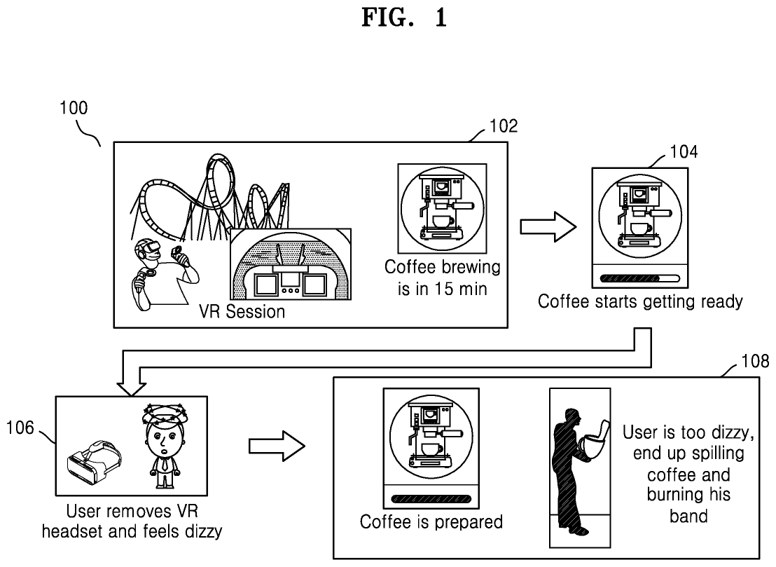

FIG. 1 depicts an example scenario 100 where an user tries to pick up freshly-brewed coffee after a VR gaming session. As depicted in operation 102, an user may be in a VR session where the user experiences high adrenaline activities which involve rapid movements. There is an upcoming activity of a real-world device; for example, a coffee machine is automated to brew coffee in 15 minutes. Later, coffee starts getting ready, as depicted in operation 104. After terminating a long VR gaming activity, when the user removes the VR headset, the user feels dizzy, as depicted in operation 106. As depicted in operation 108, the coffee is prepared by the coffee machine and the brewing smell provokes the user to grab the coffee. Since, the user is too dizzy, the user may end up spilling the coffee and burning his hand.

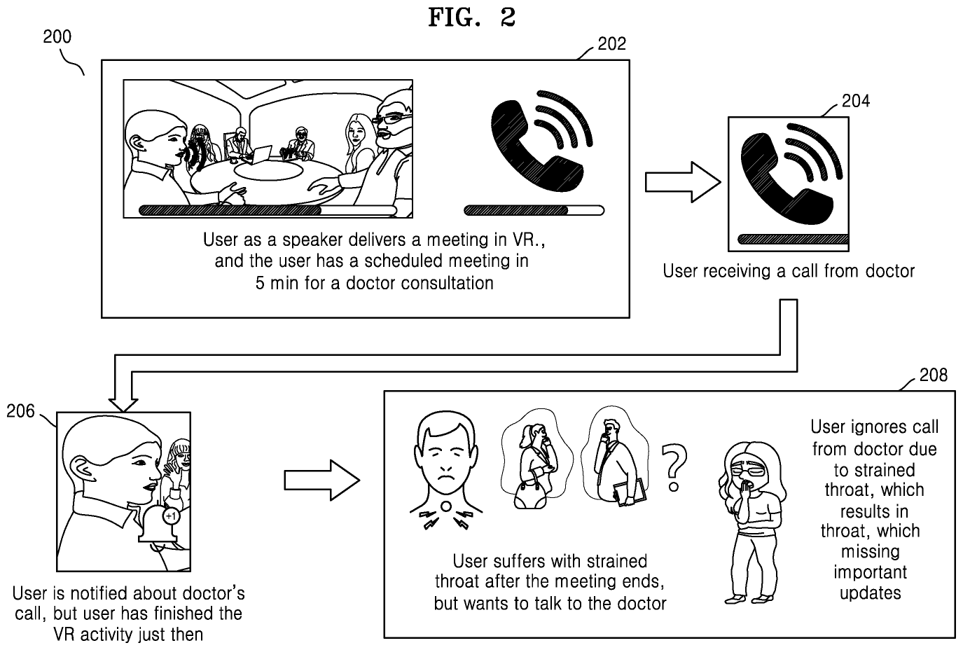

FIG. 2 depicts an example scenario 200 where an user has a scheduled call with doctor after a VR meeting. As depicted in operation 202, the user delivers the VR meeting as a speaker. The user has a scheduled meeting in 5 minutes for a doctor consultation. Later, the user receives a call from the doctor, as depicted in operation 204. The user is notified about doctor's call, but just finishes the VR activity, as depicted in operation 206. The user may have a strained throat after the meeting ends. The user ignores the call from doctor due to the strained throat which may result in missing important updates, as depicted in operation 208. Thus, after attending a prolonged VR meeting as a primary speaker, the user may feel the strained throat. The user has a scheduled call with the doctor, but due to the strained throat, the user ignores the scheduled call leading to a bad experience for the caller as well as the user.

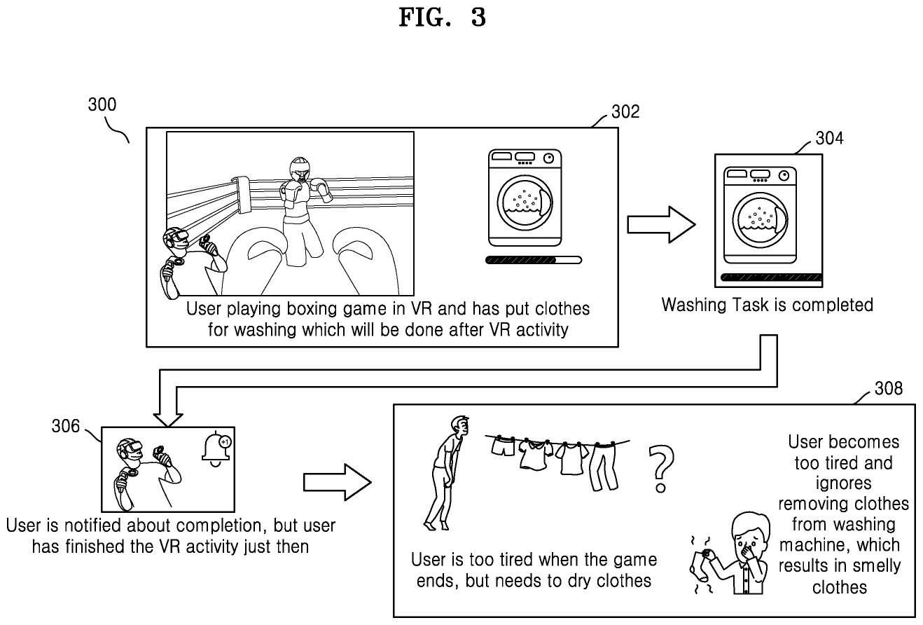

FIG. 3 depicts an example scenario 300 where an user needs to remove washed clothes after a VR boxing activity. As depicted in operation 302, the user plays a boxing game in VR after he placed clothes in a washing machine. The washing may be completed after the VR boxing activity. Later, the washing machine completes the washing task, as depicted in operation 304. The user is notified on completion of the washing task, but just finishes the VR activity then, as depicted in operation 306. The user is too tired when the game ends, but needs to hang his clothes for drying. The user does not remove the clothes from the washing machine due to tiredness, which will lead to smelly clothes, as depicted in operation 308.

As discussed above, the above systems of the related art do not identify upcoming real-world activities or associated physical efforts after terminating the VR session. Further, the systems do not correlate physical strains of one or more individual body parts of the user with the upcoming real-word activity and do not provide recommendation to the user for altering the upcoming real-world activity.

FIG. 4 depicts an electronic device 400 for managing physical exertion of an user predicted by the electronic device 400 (or determined by the electronic device 400) during a VR session. The electronic device 400 comprises at least one processor 402, a communication module 404, and a memory module 406. The electronic device 400 may be a real-world device present in a real-world environment of the user. Examples of the electronic device 400 may be, but not limited to, a desktop, a laptop, a smart phone, a personal digital assistant, a wearable device, a fitness tracker, a device configured for providing a VR environment, a gaming console, a tablet, a server, or the cloud, etc.

According to an embodiment of the disclosure, the at least one processor 402 may be configured for predicting one or more physical strains associated with one or more body parts of an user during a VR activity and creating correlation(s) between the one or more physical strains and one or more physical efforts required to perform one or more upcoming real-world activities. The at least one processor 402 may provide, to the user, one or more recommendations for altering the upcoming real-world activities. Further, the at least one processor 402 may communicate with one or more real-world devices 408 and a database 410 using the communication module 404 for processing data and mitigating repercussions of potential physical exertion predicted during a VR session, for example, in Internet of Things (JOT) environment.

According to an embodiment of the disclosure, the database 410 comprises a strain history module 422, and an upcoming data module 424. The strain history module 422 may be configured to store a historical strain data of the user of different body parts (such as eye, ear, throat, hands, legs, brain, muscles, etc.) The historical strain data may be, but not limited to, strain index, and actual time to revive from the strain. The upcoming data module 424 may be configured to store an upcoming data such as, but not limited to, at least one of an upcoming activity effort data, an upcoming user schedule, and/or upcoming IOT automations. The upcoming data may include details of upcoming activities using corresponding devices and required effort time for the activities.

The at least one processor 402 further comprises a device data aggregation module 412, a VR context managing module 414, a strain prediction module 416, a strain activity correlation module 418, and an action recommendation module 420.

According to an embodiment of the disclosure, the device data aggregation module 412 may be configured to obtain data from a plurality of real-world devices 408 as input and aggregate the input data to build an user activity data. The data from the real-world devices, such as IOT devices (such as a washing machine, a coffee machine, a gaming console, etc.), and other user devices are synchronized to a cloud storage by using an user device application. The synchronized data may be further fetched from the cloud storage whenever required.

The real-world devices 408 may be, but not limited to, VR devices (such as a VR headset), IOT devices, and wearable devices (such as a smart watch, a fitness tracker, and so on). The user activity data may be used further as input to the strain prediction module 416.

According to an embodiment of the disclosure, the VR context managing module 414 may be configured to obtain content related information from at least one of a user streamed media and type of a VR activity being performed (such as a 3D game, a meeting, an sport, and so on). The VR context managing module 414 may analyze the collected content related information to build a VR context data. The VR context managing module 414 analyses the content displayed on VR and user VR settings, such as a screen resolution, a screen refresh rate, an audio frequency, and content which user is playing, to create the VR context data. For example, if the user is in a meeting or playing a third-dimensional (3D) game etc., the VR context managing module 414 analyzes content in the VR and user VR settings and detects under which context category the analyzed content falls.

The VR context data may be used further as input to the strain prediction module 416.

According to an embodiment of the disclosure, the strain prediction module 416 may obtain the user activity data from the device data aggregation module 412. According to an embodiment of the disclosure, the strain prediction module 416 may obtain the VR context data from the VR context managing module 414. According to an embodiment of the disclosure, the strain prediction module 416 may obtain historical strain data of the user. According to an embodiment of the disclosure, the strain prediction module 416 may use the inputs (i.e., the user activity data, the VR context data, and the historical strain data of the user) to train a machine learning (ML) model. The trained ML model is utilized to predict or determine the physical strain corresponding to individual body parts of the user during the VR interaction or while the user plays the VR. The physical strain may include a strain data of some body parts, such as, but not limited to, an eye, voice, heart, hand, leg, or muscles. The VR interaction of the user may be monitored by using the VR context data obtained from the VR context managing module 414. The VR interaction may be a VR activity, such as, but not limited to, at least one of a meeting, a sport, and a game.

According to an embodiment of the disclosure, the strain activity correlation module 418 may be configured to obtain the predicted or determined physical strain data from the strain prediction module 416, and the upcoming data from the upcoming data module 424 of the database 410. The upcoming data may include the upcoming activity effort data, upcoming user schedule, and upcoming IOT automations. The strain activity correlation module 418 may create a correlation between the obtained physical strain data and the upcoming data (such as a physical effort required for performing at least one upcoming real-world activity).

The strain activity correlation module 418 takes input data such as user body strain data and upcoming data from various sources. The user body strain data may include strain on eye, ear, throat, hands, legs, brain etc. The upcoming data may include operating treadmill for 5 minutes, removing clothes from a washing machine for hanging and drying etc. The strain activity correlation module 418 creates a mapping between the upcoming data and body parts that are substantially used to do the activity. The mapped data is obtained to find the strain body part data.

The strain activity correlation module 418 may predict or determine at least one physical effort required for performing at least one upcoming real-world activity of the user, in response to (or based on) a termination of the VR session, based on the obtained data. The physical effort for performing the upcoming real-world activity may be identified by using at least one of the upcoming activity effort data, the upcoming user schedule, and the upcoming IOT automations. The similar real-world activities done in the past by the user and their respective data (such as heartrate, blood pressure, hand movement, steps taken and rest taken after completing the real-world activities) may be recorded. The recorded data may be further used to train the engine of the strain activity correlation module 418 for predicting the upcoming activity effort data.

The strain activity correlation module 418 may further identify at least one real-world device 408 through which at least one body part of the user is needed to perform the upcoming real-world activity subsequent to the VR interaction, based on the obtained determined physical strain data and the upcoming data. The upcoming real-world activity may be, but not limited to, at least one of IOT device operations, physical meetings, watching entertainment, and playing sports. The strain activity correlation module 418 may indicate whether there is a requirement for modification of the upcoming real-world activity.

According to an embodiment of the disclosure, the action recommendation module 420 may be configured to generate at least one recommendation to the user in relation to performing the upcoming real-world activity based on the correlation between the physical strain data and the upcoming data. The action recommendation module 420 may decide alternate actions, for the upcoming activities which require modification, to be recommended to the user to mitigate the repercussions of the strain based on data obtained from the strain activity correlation module 418. The recommendation may be, but not limited to, at least one of altering at least one operational setting of at least one real-world device 408 for performing the upcoming real-world activity, and delaying the upcoming real-world activity.

The action recommendation module 420 may alter the operational setting of the identified real-world device 408 for performing the upcoming real-world activity, based on the correlation. The operational setting of the real-world device 408 may be altered (for example, automatically) by sending a notification to the user via an user device and receiving an user selected response. The operational setting modification may minimize effect of the physical strain exerted on the body part of the user.

Further, the notification delivery to the user may be sent through a medium based on the strain, for example, via a speaker when eyes are strained. The action recommendation module 420 may also trigger the execution of the action selected by the user.

According to an embodiment of the disclosure, the at least one processor 402 may include one or more of microprocessors, circuits, and other hardware configured for processing. The at least one processor 402 may be configured to execute instructions stored in the memory module 406. The at least one processor 402 may be at least one of a single processer, a plurality of processors, multiple homogeneous or heterogeneous cores, multiple Central Processing Units (CPUs) of different kinds, microcontrollers, special media, and other accelerators. The at least one processor 402 may be an application processor (AP), a graphics-only processing unit such as a graphics processing unit (GPU), a visual processing unit (VPU), and/or an Artificial Intelligence (AI)-dedicated processor such as a neural processing unit (NPU).

According to an embodiment of the disclosure, the communication module 404 may be configured to enable communication between the electronic device 400 and a server through a network or a cloud network. The server may be configured or programmed to execute instructions of the electronic device 400. The communication module 404 through which the electronic device 400 and the server communicate may be in the form of either a wired network, a wireless network, or a combination thereof. The wired and wireless communication networks may include but not limited to, GPS, GSM, LAN, Wi-Fi compatibility, Bluetooth low energy (BLE) as well as NFC. The wireless communication may further comprise one or more of Bluetooth, ZigBee, a short-range wireless communication such as UWB, a medium-range wireless communication such as Wi-Fi or a long-range wireless communication such as 3G/4G or WiMAX, according to the usage environment.

According to an embodiment of the disclosure, the memory module 406 may include one or more volatile and non-volatile memory components which are capable of storing data and instructions to be executed. Examples of the memory module 406 may be, but not limited to, NAND, embedded Multi Media Card (eMMC), Secure Digital (SD) cards, Universal Serial Bus (USB), Serial Advanced Technology Attachment (SATA), solid-state drive (SSD), and so on. The memory module 406 may also include one or more computer-readable storage media. Examples of non-volatile storage elements may include magnetic hard discs, optical discs, floppy discs, flash memories, or forms of electrically programmable memories (EPROM) or electrically erasable and programmable (EEPROM) memories. In addition, the memory module 406 may, in some examples, be considered a non-transitory storage medium. The term “non-transitory” may indicate that the storage medium is not embodied in a carrier wave or a propagated signal. However, the term “non-transitory” should not be interpreted to mean that the memory module 406 is non-movable. In certain examples, a non-transitory storage medium may store data that may, over time, change (e.g., in Random Access Memory (RAM) or cache).

FIG. 4 shows example modules of the electronic device 400. According to an embodiment of the disclosure, the electronic device 400 may include less or more number of modules. Further, the labels or names of the modules are used only for illustrative purpose and does not limit the scope of the disclosure. One or more modules may be combined together to perform same or substantially similar function in the electronic device 400.

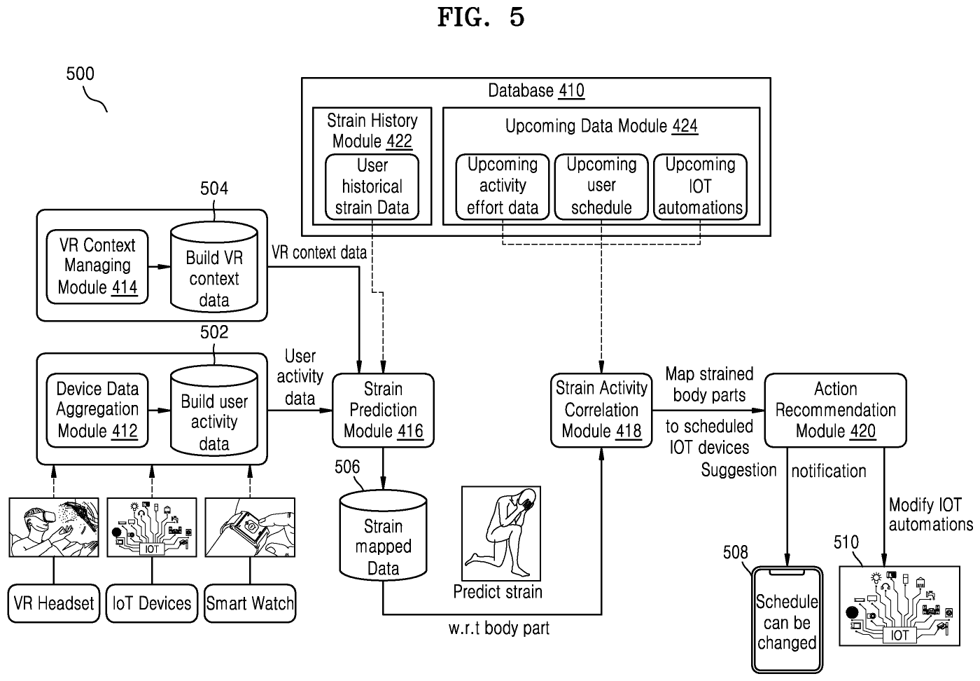

FIG. 5 depicts a system flow representation 500 for managing physical exertion of an user predicted during a VR session. The device data aggregation module 412 builds an user activity data, as depicted at operation 502, by aggregating the input data from a VR headset, IOT devices and a smart watch. The VR context managing module 414 builds a VR context data, as depicted at operation 504. The user activity data from the device data aggregation module 412, the VR context data from the VR context managing module 414, and the user historical strain data from the strain history module 422 of the database 410 may be given as input to the strain prediction module 416.

The strain prediction module 416 maps data from the input, as depicted at operation 506, and predicts (or determine) the physical strain of each body part using the ML model, during a VR interaction. The physical strain data from the strain prediction module 416 and the upcoming data from the upcoming data module 424 of the database 410 may be given as input to the strain activity correlation module 418. The upcoming data comprises upcoming activity effort data, upcoming user schedule, and upcoming IOT automations.

The strain activity correlation module 418 correlates or maps the strained body parts with the scheduled IOT devices and physical effort required to perform an upcoming real-world activity. The correlated data from the strain activity correlation module 418 is given as input to the action recommendation module 420. The action recommendation module 420 provides suggestions to the user for altering the upcoming real-world activity (for example, modifying the user schedule) by sending a notification to the user's smart device based on the correlation as depicted at operation 508. The action recommendation module 420 alters corresponding IOT device automations based on the user action in response to the notification, as depicted at operation 510.

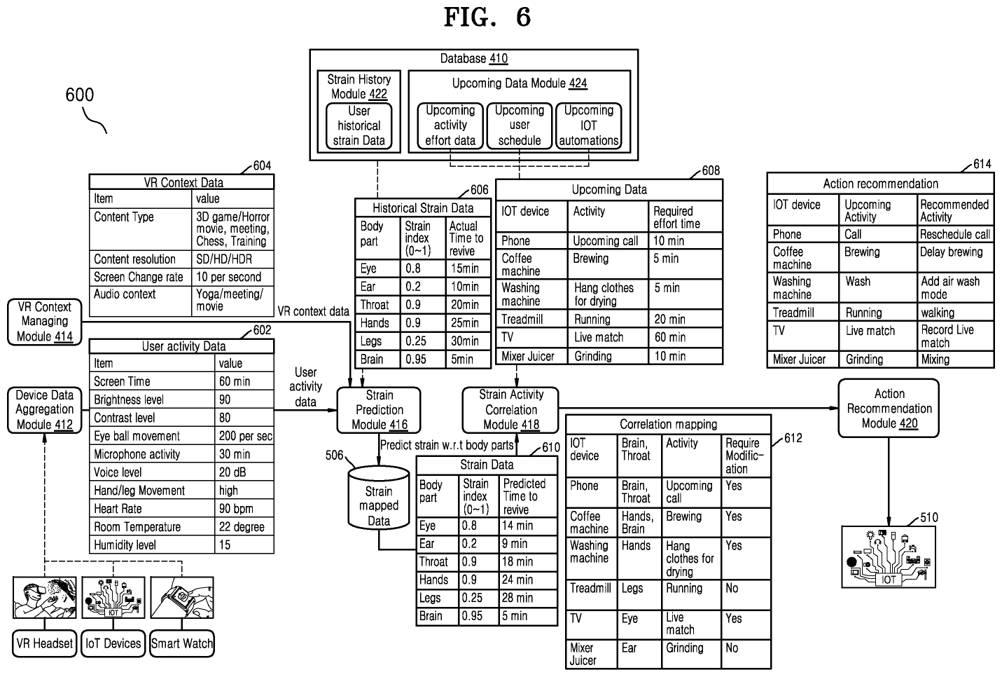

FIG. 6 depicts a detailed system flow representation 600 for managing physical exertion of an user predicted during the VR session. The user activity data built by the device data aggregation module 412 is depicted with an example table 602. For example, the device data aggregation module 412 collects input from the VR headset and builds data such as, but not limited to, screen time as 60 minutes, brightness level as 90, contrast level as 80, eyeball movement as 200 per second, microphone activity as 30 minutes, and voice level as 20 dB. The device data aggregation module 412 collects input from the smart watch and builds data such as, but not limited to, hand/leg movement as high, and heart rate as 90 bpm. The device data aggregation module 412 collects input from the IOT devices and builds data such as, but not limited to, room temperature as 22 degree, and humidity level as 15.

The VR context data built by the VR context managing module 414 is depicted with an example table 604. For example, the VR context managing module 414 obtains content related information from the user streamed media and type of the VR activity and builds data such as, but not limited to, content type as 3D game/horror movie, meeting, chess, training, content resolution as SD/HD/HDR, screen change rate as 10 per second, and audio context as yoga/meeting/movie.

The historical strain data stored in the strain history module 422 of the database 410 is depicted with an example table 606. The historical strain data may include details of strain index and actual time to revive from the strain for each body part. For example, the strain index and actual time to revive from the strain for eye is 0.8 and 15 minutes respectively, for ear is 0.2 and 10 minutes respectively, for throat is 0.9 and 20 minutes respectively, for hands is 0.9 and 25 minutes respectively, for legs is 0.25 and 30 minutes respectively, and for brain is 0.95 and 5 minutes respectively.

The upcoming data stored in the upcoming data module 424 of the database 410 is depicted with an example table 608. The upcoming data may include details of upcoming activity with corresponding IOT device and required effort time. For example, an upcoming call through phone requires effort time of 10 minutes respectively, brewing through coffee machine requires effort time of 5 minutes respectively, hanging clothes for drying from washing machine requires effort time of 5 minutes respectively, running through tread mill requires effort time of 20 minutes respectively, watching live match through TV requires effort time of 60 minutes respectively, and grinding through mixer juicer requires effort time of 10 minutes respectively.

The physical strain data is built by the strain prediction module 416 based on the user activity data, VR context data, and the historical strain data, which is depicted with an example table 610. The physical strain data may predict details of strain index and predicted time to revive from the strain for each body part. For example, the strain index and predicted time to revive from the strain for eye is 0.8 and 14 minutes respectively, for ear is 0.2 and 9 minutes respectively, for throat is 0.9 and 24 minutes respectively, for hands is 0.9 and 24 minutes respectively, for legs is 0.25 and 28 minutes respectively, and for brain is 0.95 and 5 minutes respectively.

The correlation mapping may be built by the strain activity correlation module 418 based on the predicted physical strain data and the upcoming data, which is depicted with an example table 612. The correlation mapping may identify details of upcoming activity with corresponding IOT device, required body part and whether modification is required to the upcoming activity. For example, an upcoming call through phone requires brain and throat and the activity requires modification, brewing through coffee machine requires hands and brain and the activity requires modification, hanging clothes for drying from washing machine requires hands and the activity requires modification, running through tread mill requires legs and the activity requires no modification, watching live match through TV requires eye and the activity requires modification, and grinding through the mixer juicer requires the ear and the hands and the activity requires no modification.

The recommendation data is built by the action recommendation module 420 based on the correlation mapping, which is depicted with an example table 614. The recommendation data may include details of upcoming activity with corresponding IOT device and recommended activity. For example, upcoming call through phone is recommended with reschedule call, brewing through coffee machine is recommended with delay brewing, washing clothes through washing machine is recommended with addition of air wash mode, running through tread mill is recommended with walking, watching live match through TV is recommended with record live match, and grinding through mixer juicer is recommended with mixing.

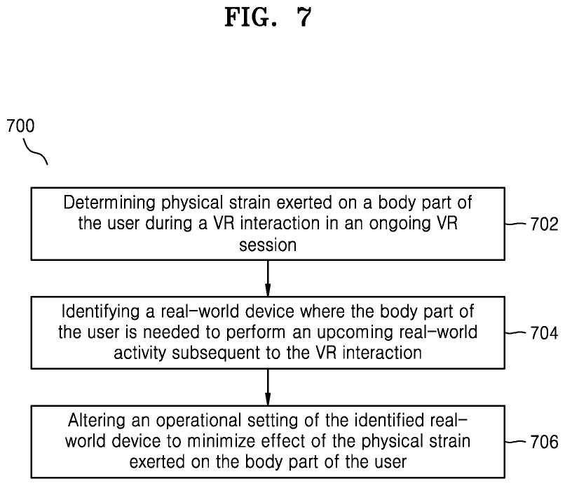

FIG. 7 depicts a method 700 for managing physical exertion of an user predicted during a VR session. The method 700 begins with determining, by the strain prediction module 416 of an electronic device 400, at least one physical strain exerted on at least one body part of the user during a VR interaction in an ongoing VR session, as depicted in operation 702. The physical strain is determined based on the user activity data, the VR context data, and the historical strain data of the user.

Subsequently, the method 700 discloses identifying, by the strain activity correlation module 418 of the electronic device 400, a real-world device 408 where the body part of the user is needed to perform an upcoming real-world activity subsequent to the VR interaction, as depicted in operation 704. The physical effort of a body part needed to perform the upcoming real-world activity by using a real-world device 408 is identified based on the determined physical strain data, an upcoming effort data, upcoming user schedule, and upcoming IOT automations.

Thereafter, the method 700 discloses altering, by the action recommendation module 420 of the electronic device 400, at least one operational setting of the identified real-world device, as depicted in operation 706. The operational setting of the real-world device 408 may be altered to minimize effect of the physical strain exerted on the body part of the user.

The above-described operations of method 700 may be performed in the order presented, in a different order or simultaneously. Further, according to an embodiment of the disclosure, some actions listed in FIG. 7 may be omitted.

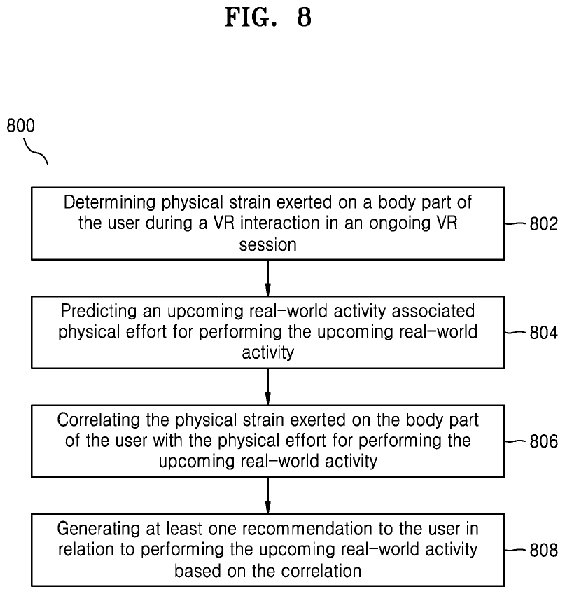

FIG. 8 depicts a method 800 for managing real-world activities of an user based on virtual world interactions of the user. The method 800 begins with determining, by the strain prediction module 416 of an electronic device 400, at least one physical strain exerted on at least one body part of the user during a VR interaction in an ongoing VR session, as depicted in operation 802. Subsequently, the method 800 discloses predicting, by the strain activity correlation module 418 of the electronic device 400, at least one physical effort for performing the upcoming real-world activity, in response to (or based on) a termination of the VR session, as depicted in operation 804.

Thereafter, the method 800 discloses correlating, by the strain activity correlation module 418 of the electronic device 400, the physical strain exerted on the body part of the user with the physical effort for performing the upcoming real-world activity, as depicted in operation 806. The correlation may be performed based on the predicted physical strain data obtained from the strain prediction module 416 and the upcoming data obtained from the database 410. Later, generating, by the action recommendation module 420 of the electronic device 400, at least one recommendation to the user in relation to performing the upcoming real-world activity, as depicted in operation 808. The recommendation may be alteration of an operating setting of a real-world device 408 or delaying the operation of the real-world device 408 by sending a notification to the user, based on the correlation.

The above-described operations of method 800 may be performed in the order presented, in a different order or simultaneously. Further, according to an embodiment of the disclosure, some actions listed in FIG. 8 may be omitted.

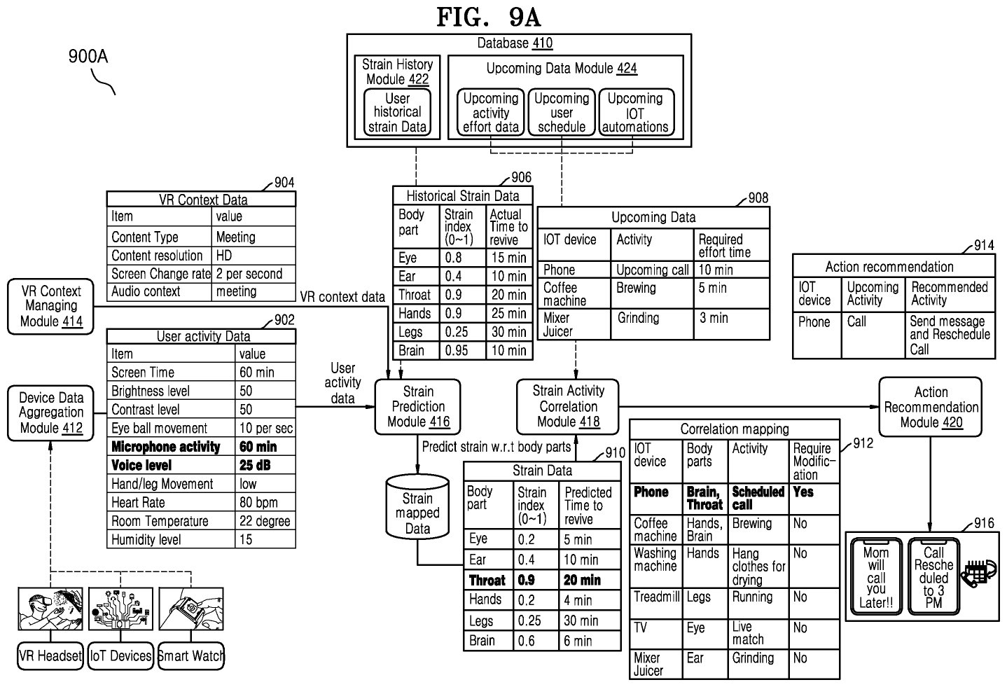

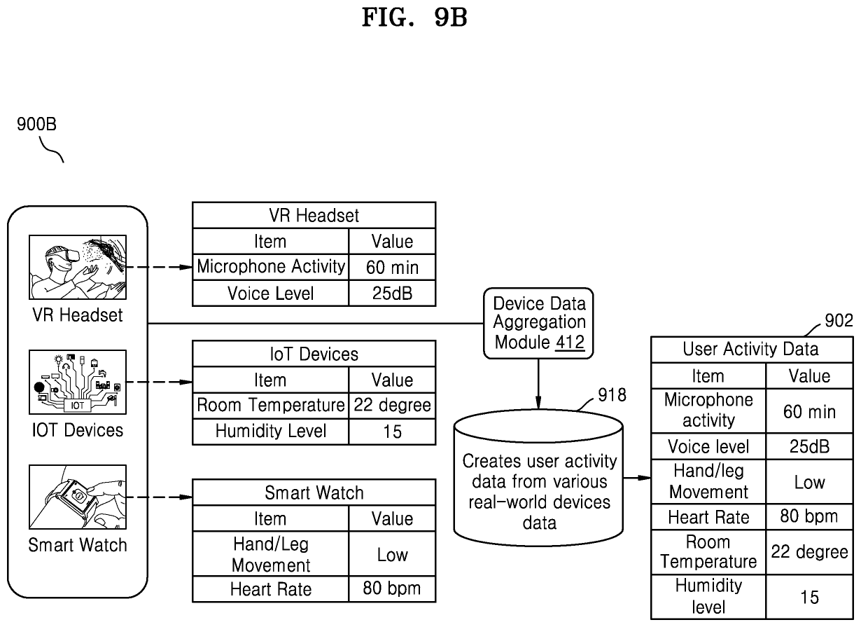

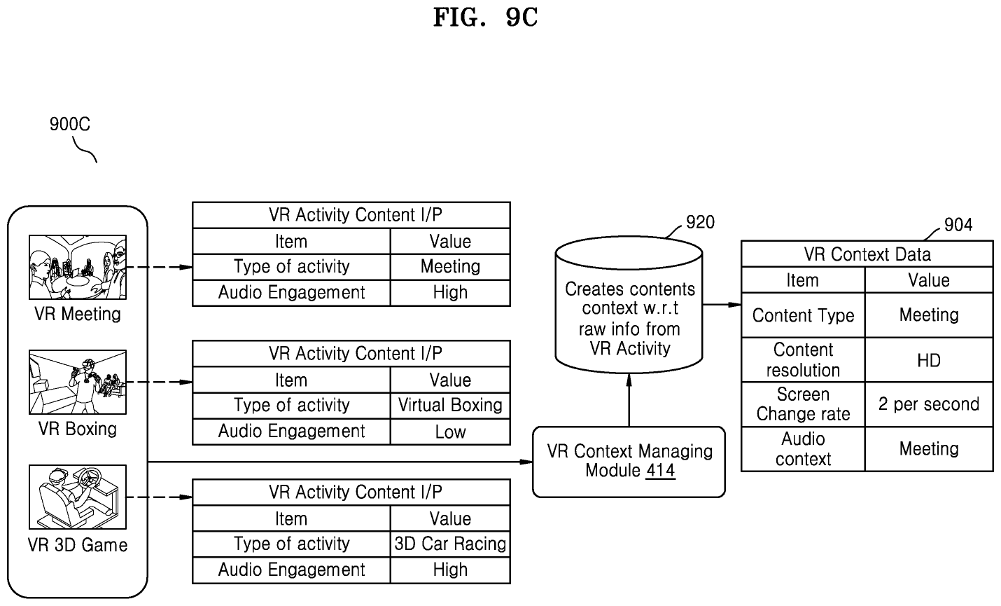

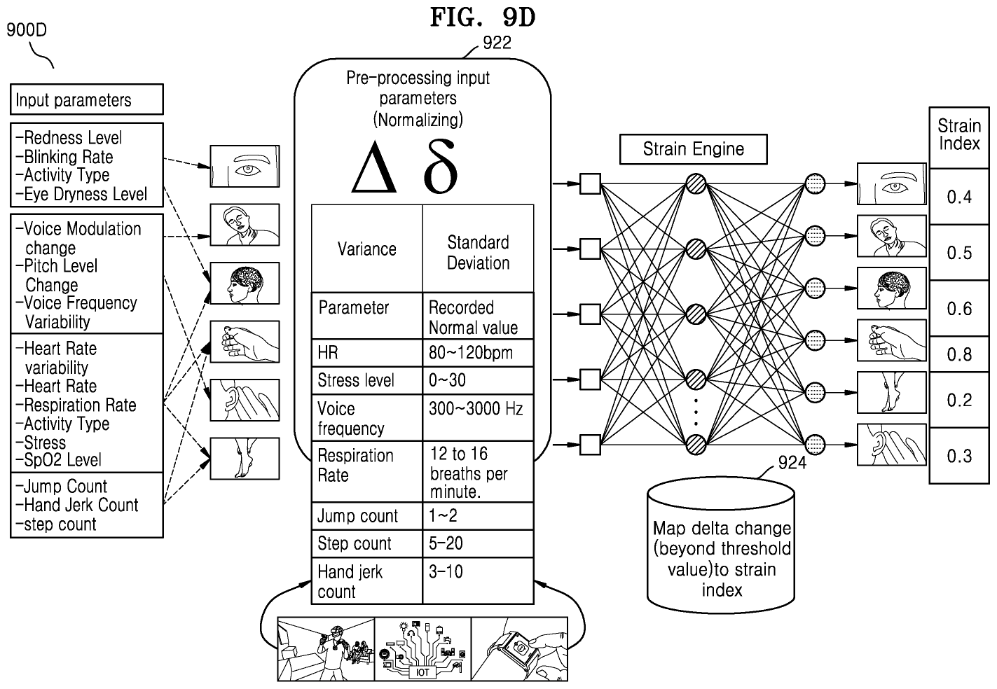

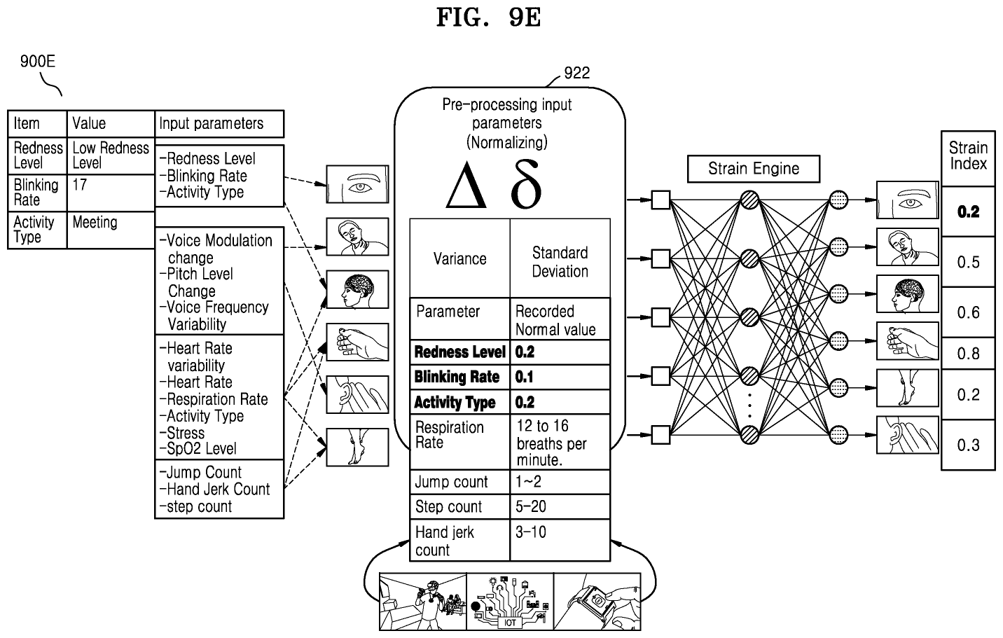

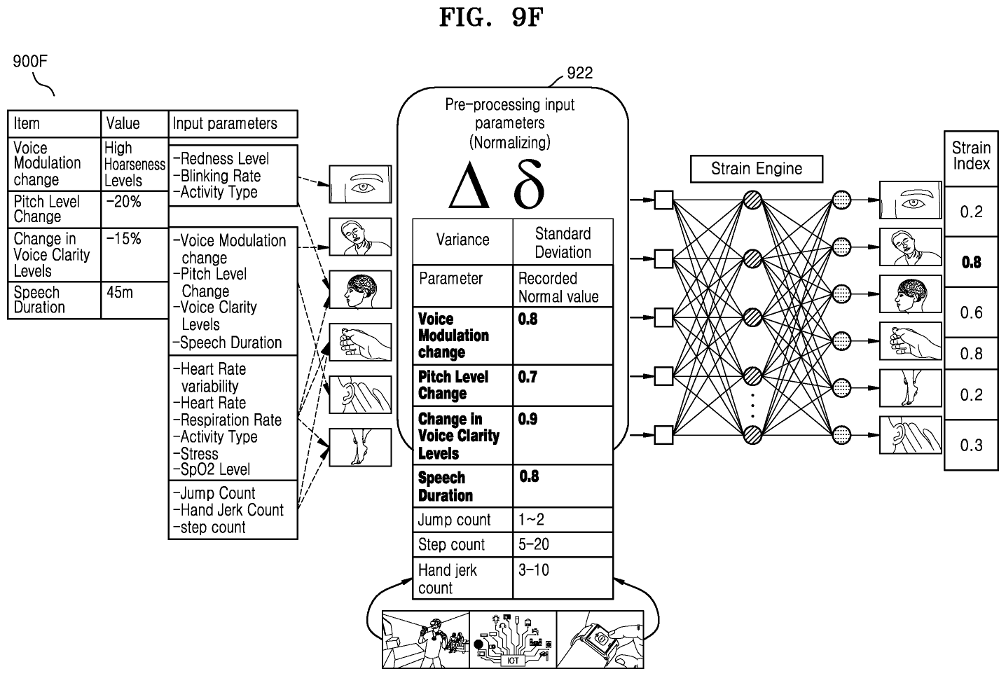

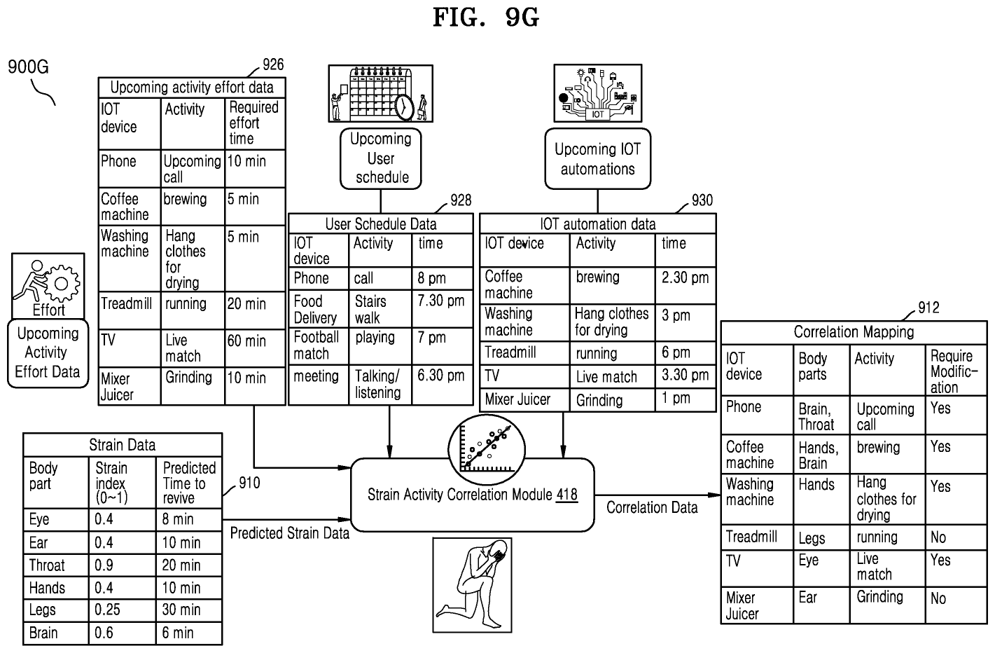

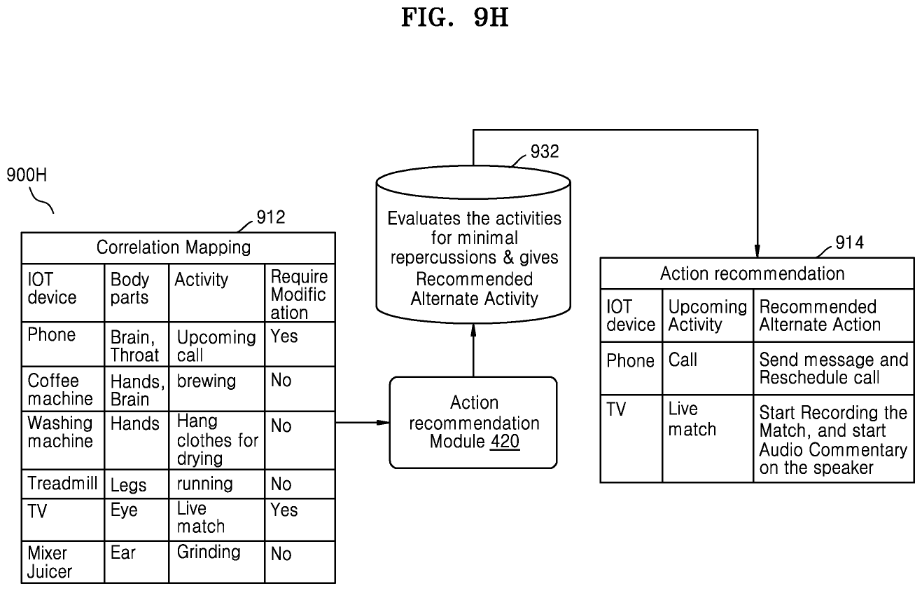

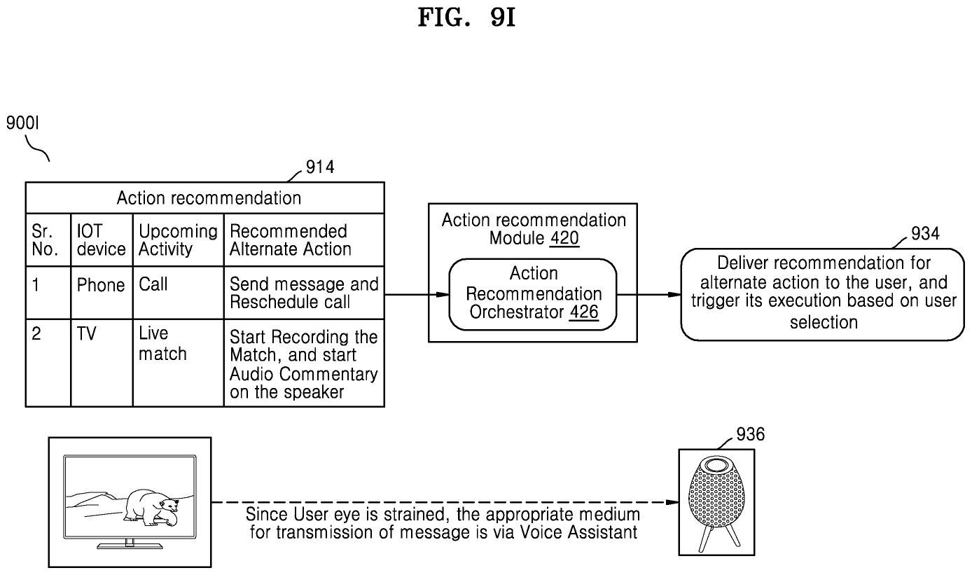

FIG. 9A depicts a use case 900A for managing physical exertion of an user by rescheduling a scheduled call after a long meeting in a VR session. The user activity data built by the device data aggregation module 412 is depicted with an example table 902. The user activity data comprises screen time as 60 minutes respectively, brightness level as 50, contrast level as 50, eyeball movement as 10 per second, microphone activity as 60 minutes respectively, voice level as 25 dB, hand/leg movement as low, and heart rate as 80 bpm, room temperature as 22 degree, and humidity level as 15. From the user activity data built by the device data aggregation module 412, a microphone activity and a voice level may include high values.

FIG. 9B depicts a detailed functional representation 900B of the device data aggregation module 412. The device data aggregation module 412 collects data from the VR headset worn by the user during a VR session. The data may include a microphone activity with 60 minutes time value and a voice level with 25 dB value, respectively. The device data aggregation module 412 collects data from the IOT devices such as a room temperature as 22 degree, and a humidity level as 15. The device data aggregation module 412 collects data from the smart watch such as a hand/leg movement as low, and a heart rate as 80 bpm. The device data aggregation module 412 creates the user activity data 902 by aggregating the collected data which includes data from the VR headset, IOT devices and the smart watch, as depicted at 918.

The VR context data built by the VR context managing module 414 is depicted with an example table 904. The VR context data built by the VR context managing module 414 may include content type as a meeting, content resolution as RD, screen change rate as 2 per second, and audio context as meeting.

FIG. 9C depicts a detailed functional representation 900C of the VR context managing module 414. The VR context managing module 414 collects content related information from an user streamed media and type of a VR activity being performed during a VR session. The VR activity content for a VR meeting may include type of activity as a meeting and an audio engagement as high. The VR activity content for VR sports (such as a VR boxing) includes type of activity as a virtual boxing and an audio engagement as low. The VR activity content for a VR 3D game may include type of activity as a 3D car racing and an audio engagement as high. The VR context managing module 414 creates VR context data 904 with respect to data from the VR activity or VR interaction, as depicted at operation 920.

The historical strain data stored in the strain history module 422 of the database 410 is depicted with an example table 906. The historical strain data may include details of strain index and actual time to revive from the strain for each body part, such as for eye is 0.8 and 15 minutes respectively, for ear is 0.4 and 10 minutes respectively, for throat is 0.9 and 20 minutes respectively, for hands is 0.9 and 25 minutes respectively, for legs is 0.25 and 30 minutes respectively, and for brain is 0.95 and 10 minutes respectively.

The upcoming data stored in the upcoming data module 424 of the database 410 is depicted with an example table 908. The upcoming data may include details of upcoming call through a phone which may require effort time of 10 minutes respectively, brewing through a coffee machine which requires effort time of 5 minutes respectively, and grinding through a mixer juicer which requires effort time of 3 minutes respectively.

The physical strain data built by the strain prediction module 416 is depicted with an example table 910. The physical strain data predicts details of strain index and predicted time to revive from the strain for each body part, such as for eye is 0.2 and 5 minutes respectively, for ear is 0.4 and 10 minutes respectively, for throat is 0.9 and 20 minutes respectively, for hands is 0.2 and 4 minutes respectively, for legs is 0.25 and 30 minutes respectively, and for brain is 0.6 and 6 minutes respectively. The strain prediction module 416 predicts strain in throat with high value based on input from the user activity data, VR context data, and the historical strain data.

FIG. 9D depicts a detailed functional representation 900D of the strain prediction module 416. The strain prediction module 416 receives input parameters of each body part from the user activity data, VR context data, and the historical strain data. The input parameters comprise redness level, blinking rate, activity type, and eye dryness level corresponding to eye and brain; voice modulation change, pitch level change, and voice frequency variability corresponding to throat and ear; heart rate variability, heart rate, respiration rate, activity type, stress, and SpO2 level corresponding to brain, hand and leg, jump count, hand jerk count, and step count corresponding to hand and leg.

The strain prediction module 416 pre-processes the input parameters by correlating the parameters with the normal recorded values using delta variance, as depicted at 922. The delta variance calculates the variance from the standard values. For example, if a normal eye blinking rate is 20 per second and user is having 10 or 30 blinks per second, then the delta variance provides the variance as 10 (shift from the normal values). Later, the delta change beyond the threshold value is mapped to strain index of each body part using the strain engine as ML model, as depicted at 924.

FIG. 9E depicts eye strain prediction 900E using the strain prediction module 416. The input values provided for eye is low redness level, blinking rate as 17, and activity type as meeting. The eye blinking rate may be captured using a computer vision algorithm. Normal blinking rate is 15-20 per minute. The blink rate patterns provide a reliable measure of individual engagement with scene content. The redness levels may be captured using the computer vision algorithm. In normal condition, the eyes may be healthy and white, and redness levels have some correlation with strain which may be obtained by classifying red and healthy eyes using deep learning. The VR activity type may determine the strain levels on the eye. The activities involving high user engagement may strain the eyes more. Such activities may include quick changing scenes such as games, and reading of small sized text compared to a meeting where main focus could be on audio. As depicted, the strain index predicted for eye is 0.2 for the values of low redness level, blinking rate as 17, and activity type as a meeting.

FIG. 9F depicts throat strain prediction 900F using the strain prediction module 416. The input values provided for throat is voice modulation change as high hoarseness levels, pitch level change as −20%, change in voice clarity levels as −15%, and speech duration as 45 minutes respectively. The voice modulation change, the pitch level change, and the change in voice clarity levels may be determined using audio analysis. The hoarseness levels of the throat indicate strained throat which causes hoarse sounds. The voice pitch reduces over long speaking cycles. As depicted, the strain index predicted for throat is 0.8 for the values of high hoarseness levels, pitch level change as −20%, change in voice clarity levels as −15%, and speech duration as 45 minutes respectively.

The correlation mapping built by the strain activity correlation module 418 is depicted with an example table 912. The correlation mapping identifies details of upcoming call through a phone which requires brain and throat and the activity requires modification, brewing through a coffee machine which requires hands and brain and the activity requires no modification, hanging clothes for drying from a washing machine which requires hands and the activity requires no modification, running through a tread mill which requires legs and the activity requires no modification, watching live match through a TV which requires eyes and the activity requires no modification, and grinding through a mixer juicer which requires ears and the activity requires no modification. Thus, the strain activity correlation module 418 identifies the scheduled call through the phone which requires modification, based on the predicted physical strain data from the strain prediction module 416 and the upcoming data obtained from the database 410.

FIG. 9G depicts a detailed functional representation 900G of the strain activity correlation module 418. The strain activity correlation module 418 obtains physical strain data, upcoming activity effort data, upcoming user schedule data, and upcoming IOT automation data, as depicted at 910, 926, 928, 930, and correlates the strained body parts and upcoming user IOT activity for suggesting whether the upcoming activity requires any modification or not. As depicted, the correlation mapping may include details of upcoming activity with corresponding IOT device, required body part and whether modification is required to the upcoming activity.

The recommendation data built by the action recommendation module 420 is depicted with an example table 914. The recommendation data may include upcoming call through phone which is recommended with sending message to user and rescheduling the call. For example, as depicted at 916, notification is sent to the user phone as “Mom will call you later!”, and “call rescheduled to 3 PM”. The action recommendation module 420 recommends an action based on the correlation mapping obtained from the strain activity correlation module 418.

FIG. 9H depicts a detailed functional representation 900H of the action recommendation module 420. The correlation mapping from the strain activity correlation module 418 is sent as input to the action recommendation module 420, and the action recommendation module 420 evaluates the activities for minimal repercussions and provides recommended alternate activity, as depicted at 932. The correlation mapping indicates as upcoming call and live match require modification. Therefore, the action recommendation module 420 recommends sending message and rescheduling call for the upcoming call; and start recording the match, and starting audio commentary on the speaker for the upcoming live match.

FIG. 9I depicts a detailed functional representation 900I of an action recommendation orchestrator 426. According to an embodiment of the disclosure, the action recommendation module 420 may include the action recommendation orchestrator 426. The action recommendation orchestrator 426 sends the alternate action recommendation output and triggers the execution of the action selected by the user, as depicted at operation 934. The action recommendation orchestrator 426 further orchestrates the notification delivery to the user through a best medium based on the strain, for example, via a voice assistant (such as the speaker 936) when eyes are strained.

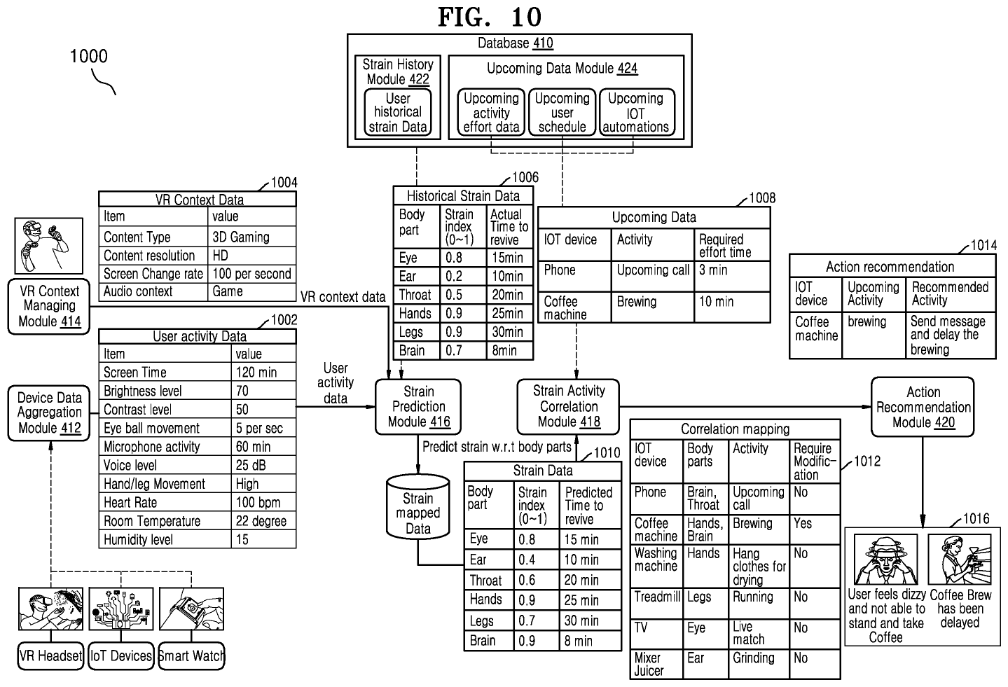

FIG. 10 depicts another use case 1000 for managing physical exertion of an user by delaying coffee preparation after a long gaming activity in a VR session. The strain prediction module 416 obtains the user activity data 1002 with screen time as 120 minutes, a brightness level as 70, a contrast level as 50, an eyeball movement as 5 per second, a microphone activity as 60 minutes, a voice level as 25 dB, hand/leg movement as high, a heart rate as 100 bpm, a room temperature as 22 degree, and a humidity level as 15. The strain prediction module 416 obtains the VR context data 1004 with content type as a 3D gaming, content resolution as HD, screen change rate as 100 per second, and audio context as a game. The strain prediction module 416 obtains the historical strain data 1006 for each body part, such as a strain index and actual time, to revive from the strain.

The strain prediction module 416 identifies the strain index and the predicted time as depicted at 1010 to revive from the strain for each body part based on the obtained data. For eyes, the strain index is 0.8 and the predicted time to revive from the strain is 15 minutes respectively. For ears, the strain index is 0.4 and the predicted time to revive from the strain is 10 minutes, respectively. For throat, the strain index is 0.6 and the predicted time to revive from the strain is 20 minutes respectively. For hands, the strain index is 0.9 and the predicted time to revive from the strain is 25 minutes, respectively. For legs, the strain index is 0.7 and the predicted time to revive from the strain is 30 minutes respectively. For brain, the strain index is 0.9 and the predicted time to revive from the strain is 8 minutes respectively. according to an embodiment of the disclosure, Hands and brain are identified with high strain values.

The strain activity correlation module 418 obtains the identified physical strain data 1010 and the upcoming data 1008. The upcoming data 1008 may include upcoming call through a phone for 3 minutes respectively and brewing through a coffee machine for 10 minutes respectively. The strain activity correlation module 418 correlates the identified physical strain data 1010 and the upcoming data 1008 and outputs the correlation mapping 1012 with modification requirement for brewing coffee which requires hands and brain involvement. The action recommendation module 420 recommends sending message and delaying the brewing of the coffee machine as depicted at 1014. As depicted at 1016, the user feels dizzy and not able to stand and take coffee, and thus, the brewing of the coffee is delayed.

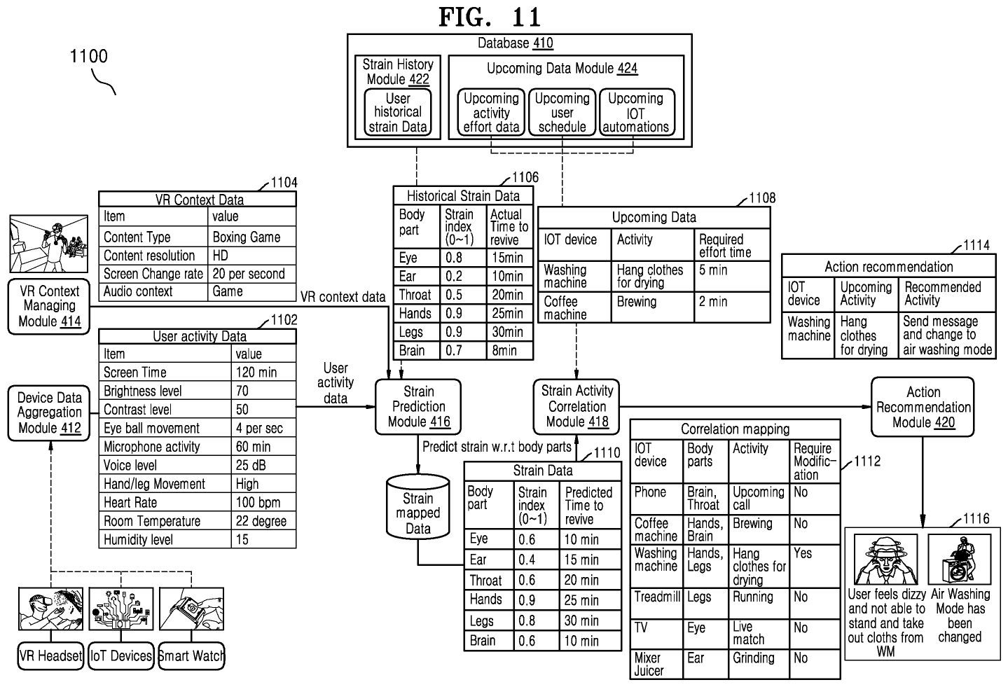

FIG. 11 depicts another use case 1100 for managing physical exertion of an user by changing a washing machine mode after a long gaming activity in a VR session. The strain prediction module 416 obtains the user activity data 1102 and the VR context data 1104 with content type as a boxing game, content resolution as HD, screen change rate as 20 per second, and audio context as a game. The strain prediction module 416 obtains the historical strain data 1106 for each body part, such as a strain index and an actual time (or predicted time) to revive from the strain.

The strain prediction module 416 identifies the strain index and the predicted time to revive from the strain for each body part, as depicted at 1110, based on the obtained data. For eyes, the strain index is 0.6 and the predicted time to revive from the strain is 10 minutes respectively. For ear, the strain index is 0.4 and the predicted time to revive from the strain is 15 minutes respectively. For throat, the strain index is 0.6 and the predicted time to revive from the strain is 20 minutes respectively. For hands, the strain index is 0.9 and the predicted time to revive from the strain is 25 minutes respectively. For legs, the strain index is 0.8 and the predicted time to revive from the strain is 30 minutes respectively. For brain, the strain index is 0.6 and the predicted time to revive from the strain is 10 minutes respectively. Hands and legs are identified with high strain values.

The strain activity correlation module 418 obtains the identified physical strain data 1110 and the upcoming data 1108. The upcoming data 1108 may include hanging clothes for drying from the washing machine for 5 minutes and brewing through the coffee machine for 2 minutes. The strain activity correlation module 418 correlates the identified physical strain data 1110 and the upcoming data 1108 and outputs the correlation mapping 1112 with modification requirement for hanging clothes for drying which requires hands and legs involvement. The action recommendation module 420 recommends sending message and changing to air washing mode as depicted at 1114. As depicted at 1116, the user feels dizzy and not able to stand and take out clothes from the washing machine, and the air washing mode has been changed.

The embodiments disclosed herein may be implemented through at least one software program running on at least one hardware device. The modules shown in FIG. 4 may include blocks which may correspond to at least one of a hardware device, or a combination of hardware device and software module.

The embodiment disclosed herein describes a device and methods to mitigate the repercussions of potential physical exertion predicted during VR session in IOT environment. Therefore, it is understood that the scope of the protection is extended to such a program and in addition to a computer readable means having a message therein, such computer readable storage means contain program code means for implementation of one or more operations of the method, when the program runs on a server or mobile device or any suitable programmable device. The method is implemented in at least one embodiment through or together with a software program written in e.g. Very high speed integrated circuit Hardware Description Language (VHDL) another programming language, or implemented by one or more VHDL or several software modules being executed on at least one hardware device. The hardware device may be any kind of portable device that may be programmed. The device may also include means which could be e.g. hardware means like e.g. an ASIC, or a combination of hardware and software means, e.g. an ASIC and an FPGA, or at least one microprocessor and at least one memory with software modules located therein. The method embodiments described herein could be implemented partly in hardware and partly in software. Alternatively, the disclosure may be implemented on different hardware devices, e.g. using a plurality of CPUs.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein may be practiced with modification within the spirit and scope of the embodiments as described herein.

According to an embodiment of the disclosure, the method may include determining at least one physical strain exerted on at least one body part of the user in at least one Virtual Reality (VR) interaction of a VR session.

According to an embodiment of the disclosure, the method may include identifying at least one real-world device where the at least one body part of the user performs at least one upcoming real-world activity subsequent to the at least one VR interaction.

According to an embodiment of the disclosure, the method may include altering at least one operational setting of the at least one identified real-world device.

According to an embodiment of the disclosure, the at least one operational setting corresponds to the at least one physical strain exerted on the at least one body part of the user.

According to an embodiment of the disclosure, the method may include monitoring the at least one VR interaction based on at least one VR context data.

According to an embodiment of the disclosure, the method may include determining the at least one physical strain based on at least one of at least one user activity data, the at least one VR context data, and at least one historical strain data of the user based on at least one of a machine learning model or indexing.

According to an embodiment of the disclosure, the method may include obtaining the at least one user activity data based on aggregated data from a plurality of real-world devices.

According to an embodiment of the disclosure, obtaining the at least one VR context data by collecting content related information from at least one of a streamed media and a type of a VR activity.

According to an embodiment of the disclosure, the method may include identifying the at least one real-world device to perform the at least one upcoming real-world activity using at least one of an upcoming activity effort data, an upcoming user schedule, and upcoming Internet of Things (IoT) automations.

According to an embodiment of the disclosure, the method may include obtaining at least one of the upcoming activity effort data, the upcoming user schedule, or the upcoming IoT automations from a database.

According to an embodiment of the disclosure, the method may include predicting at least one physical effort related to the at least one upcoming real-world activity based on a termination of the VR session.

According to an embodiment of the disclosure, the method may include correlating the at least one physical strain exerted on the at least one body part of the user with the at least one physical effort.

According to an embodiment of the disclosure, the method may include altering the at least one operational setting of the at least one real-world device for performing the at least one upcoming real-world activity, based on the correlating the at least one physical strain exerted on the at least one body part of the user with the at least one physical effort.

According to an embodiment of the disclosure, the method may include predicting the at least one physical effort for performing the at least one upcoming real-world activity based on at least one of the at least one of the upcoming activity effort data, the upcoming user schedule, or the upcoming IoT automations.

According to an embodiment of the disclosure, the method may include altering the at least one operational setting of the at least one identified real-world device by sending a notification to the user via an user device.

According to an embodiment of the disclosure, the method may include delaying the at least one upcoming real-world activity.

According to an embodiment of the disclosure, an electronic device may include at least one memory storing at least one instruction and at least one processor operatively connected to the at least one memory and configured to execute the at least one instructions.

According to an embodiment of the disclosure, at least one processor is configured to determine at least one physical strain exerted on at least one body part of the user in at least one Virtual Reality (VR) interaction of a VR session.

According to an embodiment of the disclosure, at least one processor is configured to identify at least one real-world device where the at least one body part of the user performs at least one upcoming real-world activity subsequent to the at least one VR interaction.

According to an embodiment of the disclosure, at least one processor is configured to alter at least one operational setting of the at least one identified real-world device.

According to an embodiment of the disclosure, wherein the at least one operational setting corresponds to the at least one physical strain exerted on the at least one body part of the user.

According to an embodiment of the disclosure, at least one processor is configured to monitor the at least one VR interaction based on at least one VR context data.

According to an embodiment of the disclosure, at least one processor is configured to determine the at least one physical strain based on at least one of at least one user activity data, the at least one VR context data, and at least one historical strain data of the user based on at least one of a machine learning model or indexing.

According to an embodiment of the disclosure, at least one processor is configured to obtain the at least one user activity data based on aggregated data from a plurality of real-world devices.

According to an embodiment of the disclosure, obtaining the at least one VR context data by collecting content related information from at least one of a streamed media and a type of a VR activity.

According to an embodiment of the disclosure, at least one processor is configured to identify the at least one real-world device to perform the at least one upcoming real-world activity using at least one of an upcoming activity effort data, an upcoming user schedule, and upcoming Internet of Things (IoT) automations.

According to an embodiment of the disclosure, at least one processor is configured to obtain at least one of the upcoming activity effort data, the upcoming user schedule, or the upcoming IoT automations from a database.

According to an embodiment of the disclosure, at least one processor is configured to predict at least one physical effort related to the at least one upcoming real-world activity based on a termination of the VR session.

According to an embodiment of the disclosure, at least one processor is configured to correlate the at least one physical strain exerted on the at least one body part of the user with the at least one physical effort.

According to an embodiment of the disclosure, at least one processor is configured to alter the at least one operational setting of the at least one real-world device for performing the at least one upcoming real-world activity, based on the correlating the at least one physical strain exerted on the at least one body part of the user with the at least one physical effort.

According to an embodiment of the disclosure, at least one processor is configured to predict the at least one physical effort for performing the at least one upcoming real-world activity based on at least one of the at least one of the upcoming activity effort data, the upcoming user schedule, or the upcoming IoT automations.

According to an embodiment of the disclosure, at least one processor is configured to alter the at least one operational setting of the at least one identified real-world device by sending a notification to the user via an user device.

According to an embodiment of the disclosure, at least one processor is configured to delay the at least one upcoming real-world activity.

According to an embodiment of the disclosure, at least one processor is configured to generate, to the user, at least one recommendation in relation to performing the at least one upcoming real-world activity based on the correlating the at least one physical strain exerted on the at least one body part of the user with the at least one physical effort.

What is claimed is:

1. A method performed by an electronic device for managing physical exertion of an user, comprising:
determining at least one physical strain exerted on at least one body part of the user in at least one Virtual Reality (VR) interaction of a VR session;
identifying at least one real-world device where the at least one body part of the user performs at least one upcoming real-world activity subsequent to the at least one VR interaction; and
altering at least one operational setting of the at least one identified real-world device,
wherein the at least one operational setting corresponds to the at least one physical strain exerted on the at least one body part of the user.

2. The method of claim 1, further comprising monitoring the at least one VR interaction based on at least one VR context data.

3. The method of claim 2, further comprising determining the at least one physical strain based on at least one of:
at least one user activity data,
the at least one VR context data, and
at least one historical strain data of the user based on at least one of a machine learning model or indexing.

4. The method of claim 3, further comprising:
obtaining the at least one user activity data based on aggregated data from a plurality of real-world devices; and
obtaining the at least one VR context data by collecting content related information from at least one of a streamed media and a type of a VR activity.

5. The method of claim 1, further comprising:
identifying the at least one real-world device to perform the at least one upcoming real-world activity using at least one of an upcoming activity effort data, an upcoming user schedule, and upcoming Internet of Things (IoT) automations; and
obtaining at least one of the upcoming activity effort data, the upcoming user schedule, or the upcoming IoT automations from a database.

6. The method of claim 1, wherein the altering the at least one operational setting of the at least one real-world device, comprises:
predicting at least one physical effort related to the at least one upcoming real-world activity based on a termination of the VR session;
correlating the at least one physical strain exerted on the at least one body part of the user with the at least one physical effort; and
altering the at least one operational setting of the at least one real-world device for performing the at least one upcoming real-world activity, based on the correlating the at least one physical strain exerted on the at least one body part of the user with the at least one physical effort.

7. The method of claim 6, wherein predicting at least one physical effort comprises predicting the at least one physical effort for performing the at least one upcoming real-world activity based on at least one of the at least one of the upcoming activity effort data, the upcoming user schedule, or the upcoming IoT automations.

8. The method of claim 1, further comprising altering the at least one operational setting of the at least one identified real-world device by sending a notification to the user via an user device.

9. The method of claim 1, wherein the altering of at least one operational setting of the at least one identified real-world device comprises delaying the at least one upcoming real-world activity.

10. An electronic device for managing physical exertion of an user, comprising:
at least one memory storing at least one instructions; and
at least one processor operatively connected to the at least one memory and configured to execute the at least one instructions to:
determine at least one physical strain exerted on at least one body part of the user in at least one Virtual Reality (VR) interaction of a VR session;
identify at least one real-world device where the at least one body part of the user performs at least one upcoming real-world activity subsequent to the at least one VR interaction; and
alter at least one operational setting of the at least one identified real-world device,
wherein the at least one operational setting corresponds to the at least one physical strain exerted on the at least one body part of the user.

11. The electronic device of claim 10, wherein the processor is further configured to monitor the at least one VR interaction based on at least one VR context data.

12. The electronic device of claim 11, wherein the processor is further configured to:
determine the at least one physical strain based on at least one of:
at least one user activity data,
the at least one VR context data, and
at least one historical strain data of the user based on at least one of a machine learning model or indexing.

13. The electronic device of claim 12, wherein the processor is further configured to:
obtain the at least one user activity data based on aggregated data from a plurality of real-world devices; and
obtaining the at least one VR context data by collecting content related information from at least one of a streamed media and a type of a VR activity.

14. The electronic device of claim 10, wherein the processor is further configured to:
identify the at least one real-world device to perform the at least one upcoming real-world activity using at least one of an upcoming activity effort data, an upcoming user schedule, and upcoming Internet of Things (IoT) automations; and
obtain at least one of the upcoming activity effort data, the upcoming user schedule, or the upcoming IoT automations from a database.

15. The electronic device of claim 10, wherein the processor is further configured to:
predict at least one physical effort related to the at least one upcoming real-world activity based on a termination of the VR session;
correlate the at least one physical strain exerted on the at least one body part of the user with the at least one physical effort; and
alter the at least one operational setting of the at least one real-world device for performing the at least one upcoming real-world activity, based on the correlating the at least one physical strain exerted on the at least one body part of the user with the at least one physical effort.

16. The electronic device of claim 15, wherein the processor is further configured to predict the at least one physical effort for performing the at least one upcoming real-world activity based on at least one of the at least one of the upcoming activity effort data, the upcoming user schedule, or the upcoming IoT automations.

17. The electronic device of claim 10, wherein the processor is further configured to alter the at least one operational setting of the at least one identified real-world device by sending a notification to the user via an user device.

18. The electronic device of claim 10, wherein the processor is further configured to delay the at least one upcoming real-world activity.

19. The electronic device of claim 10, wherein the processor is further configured to generate, to the user, at least one recommendation in relation to performing the at least one upcoming real-world activity based on the correlating the at least one physical strain exerted on the at least one body part of the user with the at least one physical effort.

20. A non-transitory computer-readable recording medium having recorded thereon a program for executing the method of claim 1 on a computer.
